(19) United States
(12) Patent Application Publication
(10) Pub. No.: US 2019/0209710 A1
(43) Pub. Date: Jul. 11, 2019

(54) DIAGNOSTIC SENSOR AND CHEWING GUM COMPRISING SUCH A DIAGNOSTIC SENSOR FOR THE TASTE-BASED DETECTION OF VIRUSES

(71) Applicant: Julius-Maximilians-Universitaet Wuerzburg, Wuerzburg (DE)

(72) Inventors: Lorenz MEINEL, Wuerzburg (DE); Tobias MIESLER, Wuerzburg (DE); Juergen SEIBEL, Wuerzburg (DE)

(73) Assignee: Julius-Maximilians-Universitaet Wuerzburg, Wuerzburg (DE)

(21) Appl. No.: 16/334,293

(22) PCT Filed: Aug. 28, 2017
(86) PCT No.: PCT/EP2017/071572

§ 371 Date: Mar. 18, 2019

(30) Foreign Application Priority Data
Sep. 19, 2016 (EP) 10 2016 011 298.0

(51) Int. Cl. A61K 49/00; C07H 5/06

(52) U.S. Cl. CPC A61K 49/0004; C07H 5/06

(57) ABSTRACT

The subject matter of the invention is a diagnostic sensor that can be formulated into a diagnostic chewing gum that detects relevant concentrations of influenza viruses in the oral cavity. The technical application is the initial diagnosis by those affected themselves, e.g. sore throats manifesting in them. By the detection of the influenza viruses a selective therapy of the infection is possible and employment of antibiotics that only are effective with bacterial diseases can be reduced.

### BACKGROUND OF THE INVENTION

Viral infections cause a number of diseases that burden large parts of the population and the health care system every year to a considerable extent. Above all, infections with the influenza A and B viruses in this regard concern the general public in a large scale. According to the Robert Koch-Institut two to ten millions of people fall ill with influenza every year only in Germany, wherein deaths in 2013 are estimated at 20,600 (Robert Koch-Institut, Epidemiologisches Bulletin Nr. 3 of Jan. 19, 2015). However, the mortality rates are often even higher, since many people do not directly die of the infection, but of an insufficient healing and the weakening of the immune system. Because of the non-specific symptoms such as fever, aching head and limbs, cold, cough, nausea or vomiting it is often very difficult to correctly diagnose the disease. Moreover, frequent mutations of the virus stems contribute to the fact that virulence and intensity of the symptoms often and rapidly change.

A reliable option of diagnosis contributes to a rapid and specific detection of the infection with the virus. For that, antibody-based immunoassays are often applied that identify particular epitopes of the viruses by specific binding. Moreover, a PCR-based method is available with which characteristic regions in the genotype of the virus can be detected. Both methods are disadvantageous in that they are very expensive, time-consuming, and complicated in handling, so that they are only of a small effectiveness. Moreover, immunoassays are of disadvantage in that the surfaces of influenza viruses that are partially altered by frequent mutations are no longer recognized by the assays.

Enzymatically active proteins that are specific for viruses—these are located on their envelopes—that are essential for their replication may also be used to carry out a virus detection. In that way, the influenza virus expresses a virus-specific neuraminidase that is released outward.

In nature N-acetylneuraminic acid (Neu5Ac) [1] (here indicated with numbered C atoms) is present in the glycocalyx of higher organisms terminally coupled to glycan chains and is cleaved off therefrom by neuraminidase in the process of virus release.

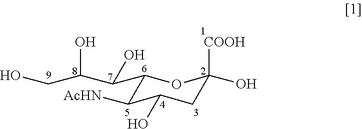 [SMILES: CC(=O)NC1C(O)CC(O)(C(=O)O)OC1C(O)C(O)CO]

Here, position 2 of [1] is the anomeric carbon atom on which the glycosidic bond to the sugar chains is. This bond is hydrolytically cleaved by neuraminidase in the course of the virus release. That is, it is logic to attach a compound that can be detected after cleavage at this site.

A method how the detection of the presence of influenza viruses can be carried out by means of the reaction of this enzyme is described in U.S. Pat. No. 5,252,458. To achieve a selective detection of only virus-related diseases the N-acetylneuraminic acid is slightly modified. In sialic acids position 4 plays an important role in the interaction between the enzyme and the substrate. It has been shown that 4-methoxy-Neu5Ac is not cut by bacterial sialidases at all, but very quickly by viral sialidases (Beau et al., Eur. J. Biochem., 106 (1980) 531-540). This differentiation is additionally enhanced by the alkylation of the hydroxyl group on position 7, so that a distinction can also be made between different virus neuraminidases. In U.S. Pat. No. 5,719,020 it is shown that 4,7-modified Neu5Ac bound to a chromogenic component is able to differentiate between influenza A and B and at the same time to differentiate bacterial from viral neuraminidases.

A similar sensor that releases a detectable dye when contacting neuraminic acid has already been marketed (ZymeTx, ZstatFlu™ Test for Influenza Types A and B). However, a disadvantage of this system is the complicated handling that like the other mentioned methods has to be performed by medical specialists. So far, only optically active systems have been connected to neuraminic acid for detection purposes.

Thus, the object of the present invention is to provide a means or method that enables the patients to detect a viral infection themselves and that enables the physician to simply differentiate a viral infection from a bacterial infection and thus, prevent the needless use of antibiotics.

According to the invention, this object is solved by a diagnostic sensor for the detection of viruses in the form of a compound of the following general formula [A]

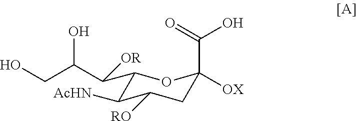

wherein the residues R independent of each other represent hydrogen or C1-C6 alkyl, preferably methyl, and the residue —OX is derived from a flavoring agent or a dye having the general formula HOX, wherein OH is a hydroxyl group and X is an aliphatic, aromatic, or heterocyclic residue.

Further, the object is solved by a diagnostic chewing gum comprising the diagnostic sensor according to the invention.

Coupling a flavoring agent is a novelty. Based on the ZstatFlu™ test we have now extended the optical application of this system in that now recognition can be by sensation of taste or odor, respectively. That is, detection is not performed outside the human body, but directly in the oral cavity by using the human tongue (sense of taste) and the nose (sense of smell) as detectors, wherein the detection in particular is by the sense of taste. When the synthesized compound contacts the neuraminidase of one of these types of viruses a flavoring agent is released that now can be perceived gustatory or olfactory, respectively.

If a dye is coupled to N-acetylneuraminic acid or a derivative thereof instead of a flavoring agent, then the detection of the viruses can also be performed in the oral cavity in case of cleavage of the coupling product by neuraminidase on the basis of a discoloration of the human tongue, i.e. by optical perception.

The diagnostic sensor according to the invention is formulated in a chewing gum and thus, touches the saliva containing neuraminidase long enough to be able to react.

Thus, the patients are able to easily diagnose themselves whether the ambiguous symptoms such as e.g. “fever” come from a bacterial or a viral infection. Such information is of importance in the subsequent treatment strategy also for the physician. By an established diagnosis of the influenza that often is wrongly treated with antibiotics it is also possible to effectively reduce the prescription of antibiotics without relevant benefit.

Additionally, the present invention relates to a method for the preparation of the diagnostic sensor and the diagnostic chewing gum according to the invention.

### SUMMARY OF THE INVENTION

In summary, the present invention relates to:
[1] A diagnostic sensor for the detection of viruses in the form of a compound of the following general formula [A]

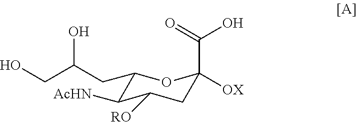

wherein the residues R independent of each other represent hydrogen or C1C6 alkyl, preferably methyl, and the residue —OX is derived from a flavoring agent having the general formula HOX, wherein OH is a hydroxyl group and X is an aliphatic, aromatic, or heterocyclic residue.
[2] A diagnostic sensor according to item [1], wherein the flavoring agent is a phenolic flavoring agent, preferably thymol or carvacrol.
[3] A diagnostic sensor according to item [1] or [2], wherein the compound of formula A is Neu5Ac-thymol having the following formula [5a]

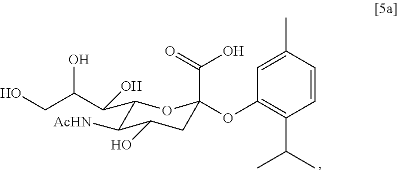 [SMILES: CC(=O)NC1C(O)CC(Oc2cc(C)ccc2C(C)C)(C(=O)O)OC1C(O)C(O)CO]

Neu5Ac-carvacrol having the following formula [5b]

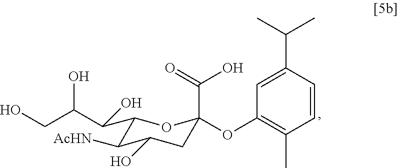 [SMILES: CC(=O)NC1C(O)CC(Oc2cc(C(C)C)ccc2C)(C(=O)O)OC1C(O)C(O)CO]

4,7-di-O-methyl-Neu5Ac-thymol having the following formula [12a]

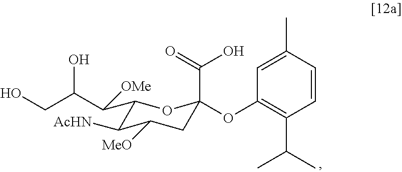 [SMILES: COC1CC(Oc2cc(C)ccc2C(C)C)(C(=O)O)OC(C(OC)C(O)CO)C1NC(C)=O]

or 4,7-di-O-methyl-Neu5Ac-carvacrol having the following formula [12b]

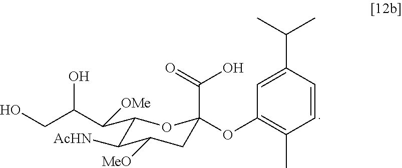 [SMILES: COC1CC(Oc2cc(C(C)C)ccc2C)(C(=O)O)OC(C(OC)C(O)CO)C1NC(C)=O]

[4] A method for the preparation of a diagnostic sensor according to any of items [1] to [3] comprising:
   (a) coupling a flavoring agent having the general formula HOX, preferably a phenolic flavoring agent, particularly preferred thymol or carvacrol, with a compound of the following formula [3]

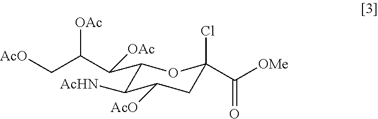

or a compound of the following formula [10]

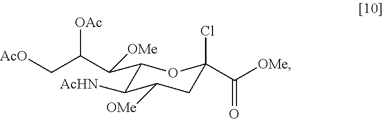 [SMILES: COC(=O)C1(Cl)CC(OC)C(NC(C)=O)C(C(OC)C(COC(C)=O)OC(C)=O)O1]

(b) deprotecting the coupling product obtained in step (a).
[5] A method according to item [4], wherein the flavoring agent in step (a) is deprotonated by a base and coupling is by nucleophilic substitution.
[6] A diagnostic chewing gum comprising the diagnostic sensor according to one of items [1] to [3].
[7] A diagnostic chewing gum according to item [6] for use in a method for the detection of viruses in a human patient.
[8] A diagnostic chewing gum for use according to item [7], wherein the detection of the viruses is in the oral cavity of the patient by optical, gustatory or olfactory perception after cleavage of the compound according to formula A and release of the flavoring agent, wherein the cleavage of compound A is by viral neuraminidase contained in the saliva of the patient.
[9] A diagnostic chewing gum for use according to item [8], wherein the viruses are influenza viruses.
[10] A diagnostic chewing gum for use according to one of items [8] and [9], wherein the detection of the viruses is by the patients themselves by means of perception of taste.
[11] A method for the preparation of a diagnostic chewing gum according to one of items [6] to [10], wherein the method comprises formulating a chewing gum with a diagnostic sensor as defined in one of items [1] to [3] or as obtained according to a method as defined in items [4] and [5].

### DETAILED DESCRIPTION OF THE INVENTION

As used in the context of the present invention, a “flavoring agent” is a substance that causes a taste-based (gustatory) or smell-based (olfactory) perception upon release in the oral cavity of a person, preferably a patient with a virus infection. Especially, thereby a taste-based perception occurs, so that the flavoring agent in particular is a tastant. The flavoring agents used in the present invention have the suitability to be used in foodstuff.

The flavoring agents used in the present invention are flavoring agents having the general formula HOX, wherein OH is a hydroxyl group and X is an aliphatic, aromatic, or heterocyclic residue.

The flavoring agents that can be used in the present invention are for example and not exhaustive acetovanillone, [alpha]-amylcinnamyl alcohol, anisyl alcohol, carveol, 4-carvomenthenol, carvacrol, cinnamyl alcohol, citronellol, dihydrocarveol, [alpha], [alpha]-dimethylphenethyl alcohol, dimethylbenzylcarbinol, 4-ethyl guaiacol, farnesol, fenchyl alcohol, 1,3,3-trimethyl-2-norbornanol, guaiacol, hydroxycitronellol, [alpha]-isobutyl phenethyl alcohol, isoeugenol, cuminyl alcohol, isopulegol, menthadienol, 2-methoxy-4-methylphenol, 2-methoxy-4-vinylphenol, methyl-5heptene-2-ol, nerol, nerolidol, 2,6-nonadiene-1-ol, phenethyl alcohol, 4-phenyl-2butanol, 4-phenyl-3-butene-2-ol, 1-phenyl-3-methyl-3-pentanol, 1-phenyl-1-propanol, 3-phenyl-1-propanol, pinocarveol, propenylguaethol, [alpha]-propylphenethyl alcohol, l-citronellol, [alpha]-terpineol, [beta]-terpineol, thymol, or verbenol.

Phenolic flavoring agents are preferred, carvacrol and thymol are particularly preferred.

In the present invention, for sake of simplicity, coupling products derived from Nacetylneuraminic acid, or a derivative thereof, are referred to as “Neu5Ac flavoring agent” or “derivative of Neu5Ac flavoring agent”. That is, a coupling product for example derived from N-acetylneuraminic acid (Neu5Ac) and thymol, in the present invention is referred to as “Neu5Ac thymol”.

The invention shows a synthesis route with which N-acetylneuraminic acid can be coupled to various flavoring agents in a quantitative yield. In combination with Nacetylneuraminic acid or its 4,7-C1-C6 alkylated derivative the flavoring agent is not or hardly gustatory perceivable by the patient. However, if the system contacts viral neuraminidase the compound is cleaved and Neu5Ac (or 4,7-(C1-C6)alkoxy-Neu5Ac) and the flavoring agent in its alcoholic OH form are formed. Now, the free flavoring agent again is able to cause gustatory and olfactory stimuli to a significant extent. Thus, a perception by the patient becomes possible, so that by the appearance of taste and smell of the selected coupled compound or by a discoloration of the tongue an influenza caused by influenza viruses can be detected.

The structure of the system that was synthesized by way of example can be characterized by the following formula [B]:

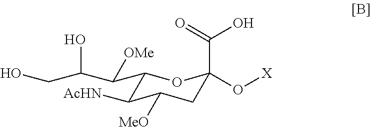

wherein —OX is derived from thymol or carvacrol. Thymol or carvacrol upon contact with the enzyme neuraminidase are cleaved off from 4,7-dimethoxy-N-acetyl-neuraminic acid and cause in the free form HO—X a sensation of taste or smell. Here, the 2-ketoside is coupled to the flavoring agent in the β-anomeric confirmation.

The compounds thymol and carvacrol are phenolic systems that sufficiently stabilize the deprotonated hydroxyl group that is required in the coupling for the nucleophilic attack on the anomeric C2 of the Neu5Ac. By our synthesis strategy it is now possible to couple the mentioned compounds to Neu5Ac or its derivatives.

N-acetylneuraminic acid bought from CarboSynth (Compton—Berkshire—UK) served as the starting material.

In the first step of the synthesis this is converted to the methyl ester. Thereby, the carboxy group in position 1 is methylated by treatment with methanol under conditions of an acidic catalysis to form product [2].

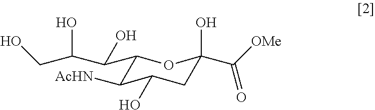 [SMILES: COC(=O)C1(O)CC(O)C(NC(C)=O)C(C(O)C(O)CO)O1]

In this step yields of 80-85% are obtained.

Subsequently, the methyl ester is treated in acetic acid with acetylchloride (AcCI), whereby on the one hand free hydroxyl groups are acetylated, but on the other hand the hydroxyl group in position 2 is converted to a chloride due to its particular reactivity as a hemiacetal. Due to the reactivity and instability processing of the obtained product [3] of this reaction is continued immediately.

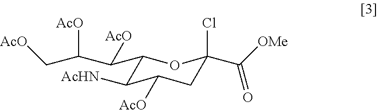 [SMILES: COC(=O)C1(Cl)CC(OC(C)=O)C(NC(C)=O)C(C(OC(C)=O)C(COC(C)=O)OC(C)=O)O1]

Now, the compound to be coupled is deprotonated with sodium hydride (NaH), so that it can nucleophilically attack on the activated C2 of the compound. In the absence of atmospheric oxygen (with a protective gas N₂) now the coupling reaction of N-acetyl-neuraminic acid and the selected flavoring agent can proceed. Under these conditions, coupling with thymol resulted in yields of ca. 30%. Thereby, the following product [4a] was formed:

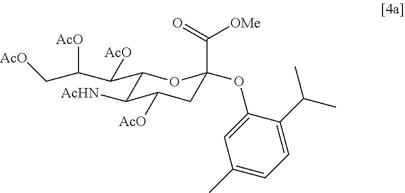 [SMILES: COC(=O)C1(Oc2cc(C)ccc2C(C)C)CC(OC(C)=O)C(NC(C)=O)C(C(OC(C)=O)C(COC(C)=O)OC(C)=O)O1]

After cleavage of the protective groups in positions 4, 7, 8, 9 by alkaline treatment of the compound, e.g. by aqueous NaOH, and saponification of the methyl ester the following final product [5a] is formed:

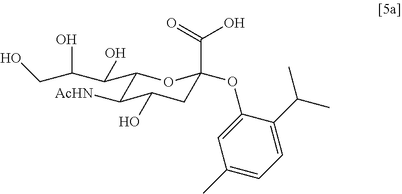 [SMILES: CC(=O)NC1C(O)CC(Oc2cc(C)ccc2C(C)C)(C(=O)O)OC1C(O)C(O)CO]

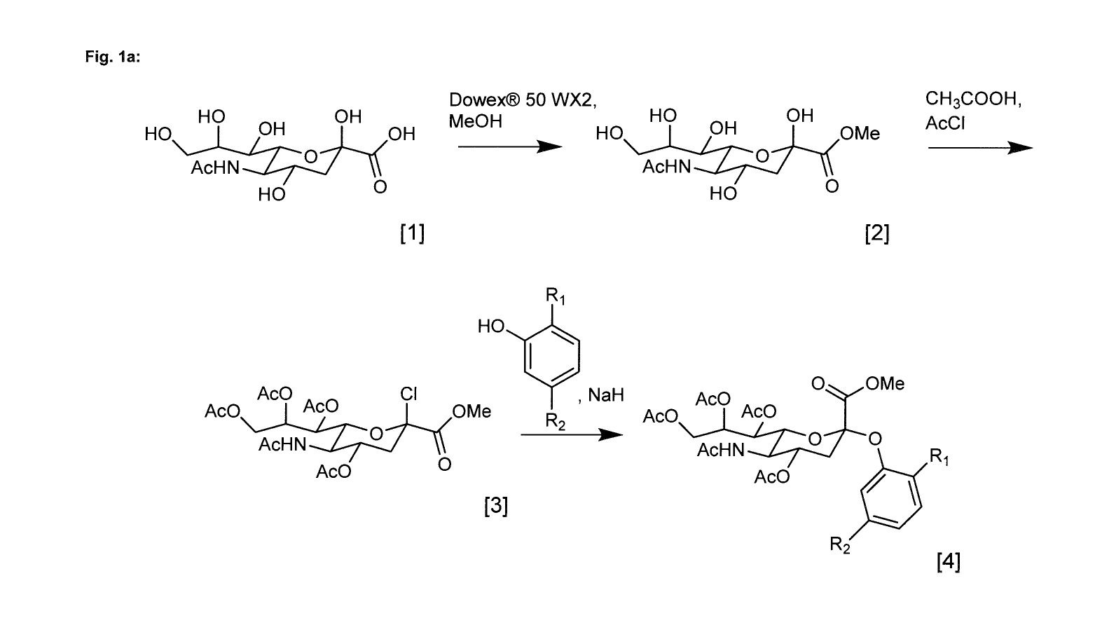

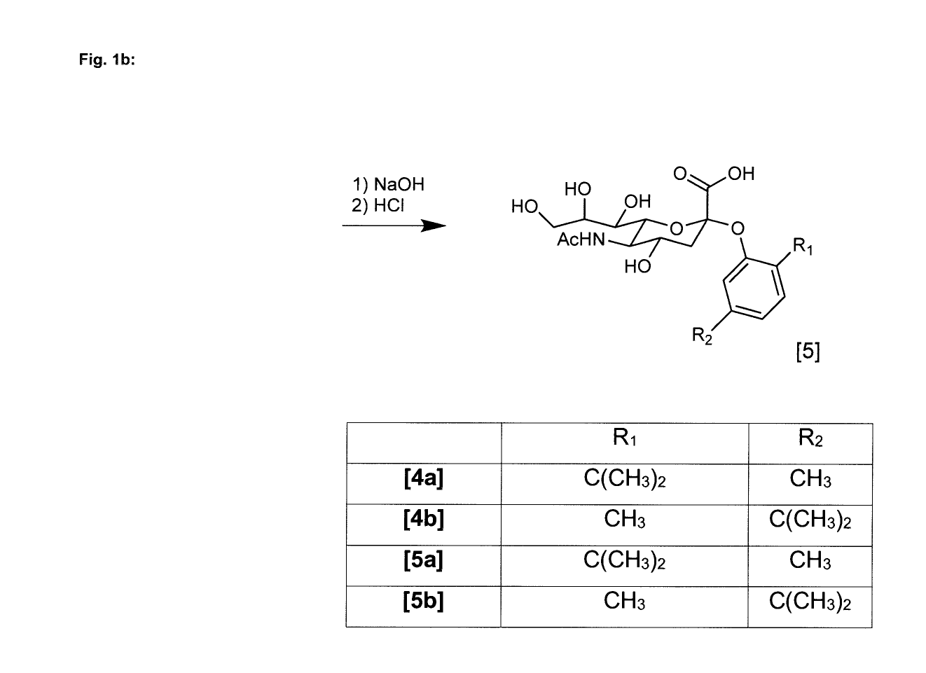

In the present case, Neu5Ac was coupled to thymol. A further possible coupling component is the isomer of the thymol carvacrol that also is well perceptible in taste and in which the isopropylidene group and the methyl group are exchanged compared to thymol. Because in this the steric hindrance is lower, but otherwise reactivity is similar, this compound can also be coupled with the synthesis scheme used with thymol. The synthesis strategy of Neu5Ac-thymol and Neu5Ac-carvacrol is summarized in FIG. 1.

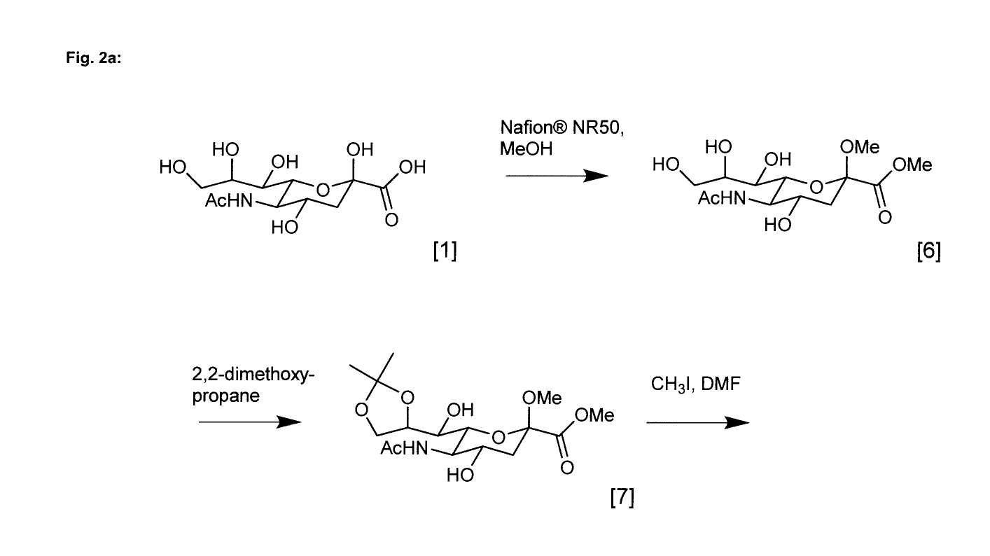

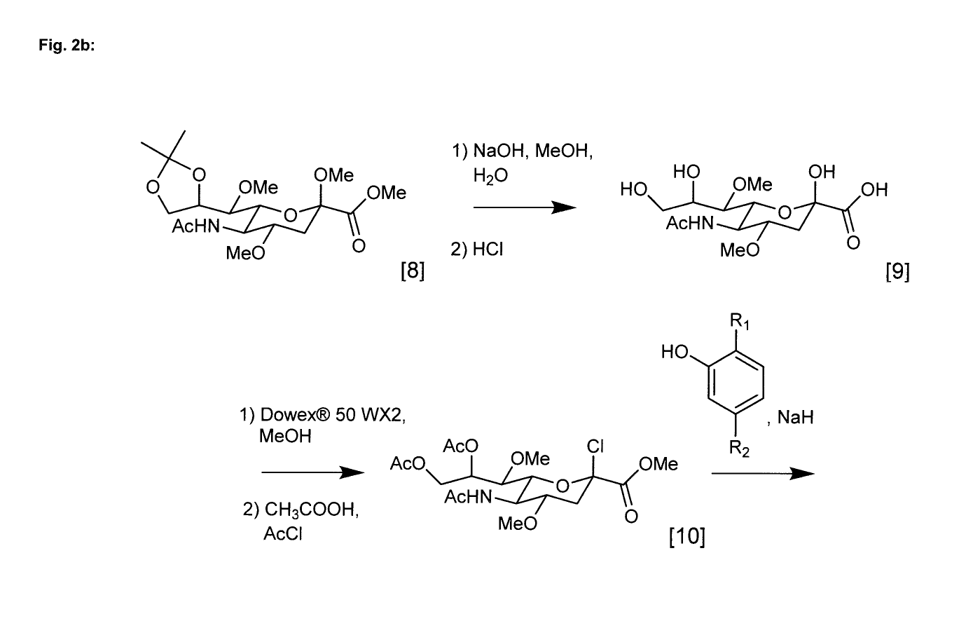

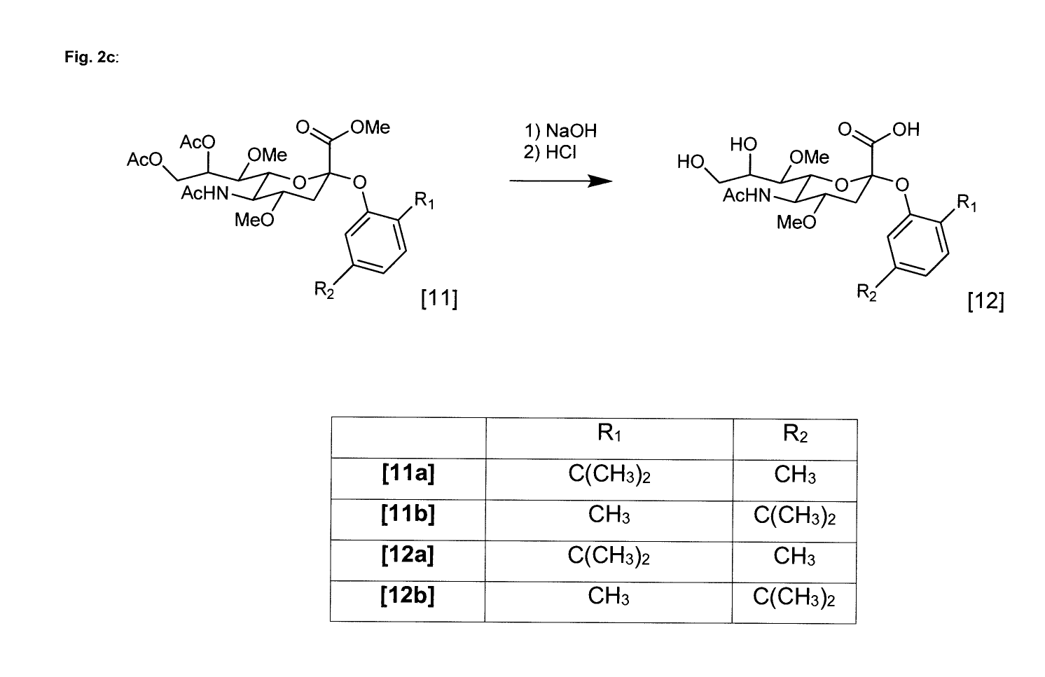

In the coupling of the flavoring agent with the 4,7-di-O-methylated derivative first the methyl groups have to be inserted at the Neu5Ac before both components can be coupled. In U.S. Pat. No. 6,303,764 B1 it is dealt in detail with the synthesis of a compound suitable for coupling and it is carried out as described there. Only the last step, the coupling with the now 4,7-di-O-methylated Neu5Ac, is changed, so that our compounds can be coupled. The synthesis strategy is schematically set forth in FIG. 2, wherein component [10] described in the publications is coupled with the flavoring agents we used. In previous publications and patents the unchanged and mono- and di-methylated Neu5Ac has been coupled to a number of fluorogenic and chromogenic components. There were no significant differences among the various Neu5Ac derivatives as to the reactivity in the coupling. In order to achieve a selective coupling the Koenigs-Knorr method is applied to the di-methylated compound. Here, the phenolic group of the flavoring agent is coupled to the Neu5Ac derivative by means of silver carbonate. Thereby, first a glycosyl chloride [10] is formed by reaction with acetyl chloride as with the non-methylated variation, wherein at the same time the remaining hydroxyl groups are again provided with acetyl protective groups.

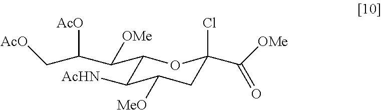 [SMILES: COC(=O)C1(Cl)CC(OC)C(NC(C)=O)C(C(OC)C(COC(C)=O)OC(C)=O)O1]

By reacting the glycosyl chloride with silver carbonate a dioxolanium ion is finally formed that can be attacked by an alcoholic group. Thereby, the following product [11] is formed:

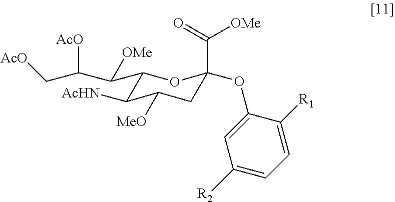

By deprotection with catalytic amounts of sodium methanolate in methanol the non-protected final product [12] is formed:

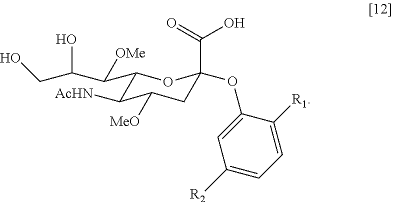

Thus, we request patent protection not only for compounds Neu5Ac-thymol [5a] and Neu5Ac-carvacrol [5b], but also for compounds 4,7-di-O-methyl-Neu5Ac-thymol [12a] and 4,7-di-O-methyl-Neu5Ac-carvacrol [12b] derived therefrom.

The invention is illustrated by the following examples.

### Example 1: Synthesis of Neu5Ac-methyl ester [2]

1.563 g of Neu5Ac [1] (CarboSynth) are dispersed in 50 ml of anhydrous methanol. 2 g of Dowex 50 W×2 (washed with dry methanol) are added and the batch is stirred at room temperature for 5 hours. Thereafter, the ion exchanger is filtered off and washed several times with methanol. After having removed the methanol by distillation product [2] is purified with silica gel (eluent:methanol) to obtain 1.354 g (83%) of [2].

### Example 2: Synthesis of Neu5Ac-thymol [5a]

Acetyl chloride (10 ml) is added to a solution of the methyl ester of Neu5Ac [2] (603 mg, 1.95 mmol) in acetic acid (10 ml) and the batch is left sealed at room temperature for 18 hrs under stirring. Subsequently, the solution is evaporated under vacuum and dried by adding toluene to obtain [3] as a white residue. The product is used without further purification due to the instability of the chloride.

A solution of thymol (510 mg, 3.40 mmol) in dimethylformamide (DMF) (10 ml) is slowly added dropwise to a suspension of NaH (60% in mineral oil, 82 mg, 3.429 mmol) in tetrahydrofurane (THF) at 0° C. and the reaction mixture is stirred for 10 min. To the blend is added the solution of the chloride [3] in THF within 15 minutes. After complete addition the mixture is stirred for 19 hours at room temperature. Thereafter, the mixture is diluted with 40 ml of ethyl acetate and transferred to a separatory funnel. Now, the organic phase is washed with water (2×30 ml) and saturated sodium chloride solution (2×30 ml), dried over Na₂SO₄, and the solvent is removed under reduced pressure.

The residue (containing [4a]) is dissolved in methanol (8 ml), 0.1M of NaOH (2 ml) is added, and stirred for one hour at room temperature. The reaction batch is brought to pH=4.5-5 with 3M HCl and extracted with dichloromethane (DCM) (3×15 ml). The aqueous phase is purified with a FPLC system (Äkta purifier, GE Healthcare) using reversed phase chromatography (RPC) on a C18 column (Phenomenex®) (eluent A: 0.1% trifluoroacetic acid (TFA) in water, eluent B: 0.1% TFA in acetonitrile). Subsequent freeze drying results in N-acetylneuraminic acid-thymol [5a] as a white powder. The total yield is 253 mg (29.4%).

### Example 3: Synthesis of 4,7-di-O-methyl-Neu5Ac-thymol [12a]

Acetyl chloride (3 ml) is added to a solution of the methyl ester of 4,7-di-O-methyl-Neu5Ac [9a] (33 mg, 0.094 mmol) in acetic acid (3 ml) and the batch is left sealed at room temperature for 18 hrs under stirring. Subsequently, the solution is evaporated under vacuum and dried by adding toluene to obtain [3] as a white residue. The product is used without any further purification due to the instability of the chloride.

The beta-silyl chloride [10a] is dissolved in 10 ml of anhydrous DCM. In a further flask, 586 mg of thymol (3.9 mmol), 95 mg of silver carbonate (0.345 mmol), and 300 mg of molecular sieve 4 Å are dissolved in 10 ml anhydrous DCM in the absence of light and oxygen. The first solution with the educt is slowly added to the second one and stirred for 72 h at room temperature.

Thereafter, the mixture is diluted with 40 ml of ethyl acetate and transferred to a separatory funnel. Now, the organic phase is washed with water (2×30 ml) and saturated sodium chloride solution (2×30 ml), dried over Na₂SO₄, and the solvent is removed under reduced pressure. The residue (containing [11a]) is dissolved in methanol (8 ml), 0.1M sodium methanolate is added up to a pH of 8-9, and stirred for one hour at room temperature. The reaction batch is neutralized with 1M HCl, filtered, and extracted with dichloromethane (DCM) (3×15 ml). The aqueous phase is purified with a FPLC system (Äkta purifier, GE Healthcare) using reversed phase chromatography (RPC) on a C18 column (Phenomenex®) (eluent A: 0.1% trifluoroacetic acid (TFA) in water, eluent B: 0.1% TFA in acetonitrile). Subsequent freeze drying results in 4,7-di-O-methyl-Neu5Ac-thymol [12a] as a white powder. The total yield is 10.2 mg (23.1%).

1. A diagnostic sensor for the detection of viruses in the form of a compound of the following general formula [A] 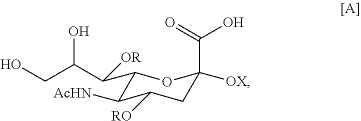
wherein each R residues is independently a hydrogen or a C1-C6 alkyl, and the residue —OX is derived from a flavoring agent having the general formula HOX, wherein OH is a hydroxyl group and X is an aliphatic, aromatic, or heterocyclic residue.

2. The diagnostic sensor according to claim 1, wherein the flavoring agent is a phenolic flavoring agent.

3. The diagnostic sensor according to claim 1, wherein the compound of formula A is selected from the group consisting of:
Neu5Ac-thymol having the following formula [5a] 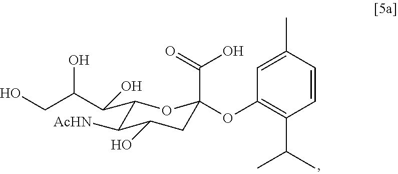 [SMILES: CC(=O)NC1C(O)CC(Oc2cc(C)ccc2C(C)C)(C(=O)O)OC1C(O)C(O)CO]
Neu5Ac-carvacrol having the following formula [5b] 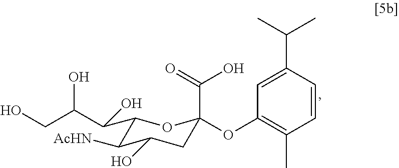 [SMILES: CC(=O)NC1C(O)CC(Oc2cc(C(C)C)ccc2C)(C(=O)O)OC1C(O)C(O)CO]
4,7-di-O-methyl-Neu5Ac-thymol having the following formula [12a] 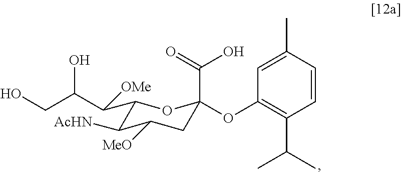 [SMILES: COC1CC(Oc2cc(C)ccc2C(C)C)(C(=O)O)OC(C(OC)C(O)CO)C1NC(C)=O]
and
4,7-di-O-methyl-Neu5Ac-carvacrol having the following formula [12b]

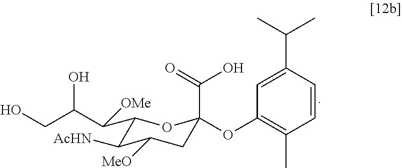 [SMILES: COC1CC(Oc2cc(C(C)C)ccc2C)(C(=O)O)OC(C(OC)C(O)CO)C1NC(C)=O]

4. A method for the preparation of a diagnostic sensor according to claim 1, said method comprising the steps of:
(a) coupling a flavoring agent having the general formula HOX with a compound of the following formula [3] 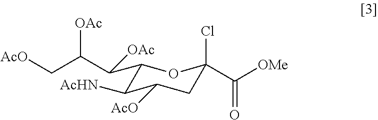
or a compound of the following formula [10] 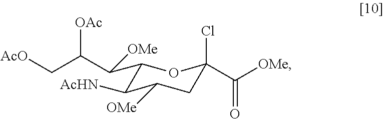 [SMILES: COC(=O)C1(Cl)CC(OC)C(NC(C)=O)C(C(OC)C(COC(C)=O)OC(C)=O)O1]
and
(b) deprotecting the coupling product obtained in step (a).

5. A diagnostic chewing gum comprising the diagnostic sensor according to claim 1.

6. A method for the detection of viruses in a human patient comprising the step of administering a diagnostic chewing gum comprising the diagnostic sensor according to claim 1 to said patient and using said diagnostic sensor to detect the presence of viruses in said patient.

7. The method according to claim 6, wherein the viruses are detected in the oral cavity of the patient by gustatory or olfactory perception after cleavage of the compound according to formula A and release of the flavoring agent, wherein the cleavage of compound A is by viral neuraminidase contained in the saliva of the patient.

8. The method according to claim 7, wherein the viruses are influenza viruses.

9. The method according to claim 8, wherein the detection of the viruses is by the patients themselves by means of perception of taste.

10. A method for the preparation of a diagnostic chewing gum comprising the diagnostic sensor according to claim 1, wherein the method comprises the step of formulating a chewing gum with a diagnostic sensor as defined in claim 1.

11. A method for the preparation of a diagnostic chewing gum comprising a diagnostic sensor for the detection of viruses in the form of a compound of the following general formula [A] 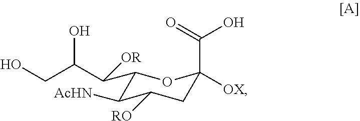
wherein each R residue is independently a hydrogen or a C1-C6 alkyl, and the residue —OX is derived from a flavoring agent having the general formula HOX,
wherein OH is a hydroxyl group and X is an aliphatic, aromatic, or heterocyclic residue,
wherein the method comprises the step of formulating a chewing gum with a diagnostic sensor obtained according to a method as defined in claim 4.

12. The diagnostic sensor according to claim 1, wherein each R is methyl.

13. The diagnostic sensor according to claim 2, wherein the phenolic flavouring agent is either thymol or carvacrol.

14. The method according to claim 4, wherein the flavoring agent having the general formula HOX is a phenolic flavouring agent.

15. The method according to claim 14, wherein the phenolic flavouring agent is either thymol or carvacrol.
